(19) United States
(12) Patent Application Publication
(10) Pub. No.: US 2017/0027468 A1
(43) Pub. Date: Feb. 2, 2017

(54) Electrocardiogram Detector

(71) Applicant: KAOHSIUNG VETERANS GENERALHOSPITAL, Kaohsiung City (TW)

(72) Inventors: Wei-Chun Huang, Kaohsiung City (TW); Cheng-Chung Hung, Kaohsiung City (TW); Hsin-Ju Huang, Kaohsiung City (TW); Chien-Ju Huang, Kaohsiung City (TW); Tzu-Wen Lin, Kaohsiung City (TW); Jin-Shiou Yang, Kaohsiung City (TW); Shue-Ren Wann, Kaohsiung City (TW); Guang-Yuan Mar, Kaohsiung City (TW); Chun-Peng Liu, Kaohsiung City (TW)

(21) Appl. No.: 15/223,026

(22) Filed: Jul. 29, 2016

(30) Foreign Application Priority Data
Jul. 31, 2015 (TW) 104124929

(51) Int. Cl. A61B 5/0408

(52) U.S. Cl. CPC A61B 5/04085; A61B 5/04087

(57) ABSTRACT

An electrocardiogram detector including a pad, an electrical connection unit, a plurality of retaining members, and a plurality of electrode pieces is disclosed. The pad has a first face, a second face, and a plurality of through-holes. The electrical connection unit has an electrical connection port and a plurality of conducting lines electrically connected to the electrical connection port. The plurality of conducting lines is arranged on the first face of the pad. The plurality of retaining members is fixed to the first face of the pad and electrically connected to the plurality of conducting lines. The plurality of electrode pieces is electrically connected to the plurality of retaining members. The plurality of electrode pieces is fixed by the plurality of retaining members. In this arrangement, convenient operation of the electrocardiogram detector is improved.

### CROSS REFERENCE TO RELATED APPLICATIONS

The application claims the benefit of Taiwan application serial No. 104124929, filed on Jul. 31, 2015, and the subject matter of which is incorporated herein by reference.

### BACKGROUND OF THE INVENTION

1. Field of the Invention

The present disclosure generally relates to an electrocardiogram detector and, more particularly, to an electrocardiogram detector that can be placed on a certain part of the body of the user for detecting the electrocardiogram signals.

2. Description of the Related Art

Electrocardiography is a main process to determine the functioning of the heart of the user. During the electrocardiogram (ECG) detection, a plurality of electrodes needs to be placed on certain body parts of the user to attain an accurate detection.

FIG. 1 shows a conventional electrocardiogram detector 9 (which is called “ECG detector 9” hereinafter) that is used to detect a twelve-lead electrocardiogram. The ECG detector 9 includes a pad 91 having a plurality of through-holes 92. The ECG detector 9 has a reference line L. During the detection, the pad 91 can be placed on the chest of the user, and the reference line L can be aligned with the midsternal line of the user. In this arrangement, the through-holes 92 can indicate the locations of the body parts to which the electrode plates E should be placed, attaining a proper placement of the electrode plates E on the body of the user.

In the ECG detector 9, although the through-holes 92 can indicate the locations of the body parts on which the electrode plates E should be placed, there are no retaining members provided at the through-holes 92 for retaining the electrode plates E. In addition, with the absence of the retaining members, the electrode plates E cannot be fixed to the surface of the pad 91 via the retaining members. In a case where the ECG detector 9 does not include the electrode plates E, it is impossible for the ECG detector 9 to detect the ECG signals simply by placing the ECG detector 9 on the chest of the user. Instead, the ECG detector 9 requires the electrode plates E to be disposed on the body of the user one by one. Therefore, the operation of the ECG detector 9 is inconvenient.

In light of the disadvantage of the conventional ECG detector 9, it is necessary to provide an electrocardiogram detector with convenient operation.

### SUMMARY OF THE INVENTION

It is therefore the objective of this disclosure to provide an electrocardiogram detector that is convenient to operate.

In an embodiment of the disclosure, an electrocardiogram detector is disclosed. The electrocardiogram detector includes a pad, an electrical connection unit, a plurality of retaining members, and a plurality of electrode pieces. The pad has a first face, a second face, and a plurality of through-holes extending through the pad from the first face to the second face. The electrical connection unit has an electrical connection port and a plurality of conducting lines electrically connected to the electrical connection port. The plurality of conducting lines is arranged on the first face of the pad. The plurality of retaining members is fixed to the first face of the pad and electrically connected to the plurality of conducting lines. Each of the plurality of retaining members includes a fixing portion. Each of the fixing portions of the plurality of retaining members forms a space in communication with a respective one of the plurality of through-holes. The plurality of electrode pieces is electrically connected to the plurality of retaining members. Each of the plurality of electrode pieces includes a connection portion and a detection portion. Each of the connection portions of the plurality of electrode pieces extends through a respective one of the plurality of through-holes and is engaged with the fixing portion of a respective one of the plurality of retaining members.

In another embodiment of the disclosure, an electrocardiogram detector is disclosed. The electrocardiogram detector includes a pad, an electrical connection unit, a plurality of retaining members, and a plurality of electrode pieces. The pad has a first face and a second face. The pad includes a plurality of through-holes extending through the pad from the first face to the second face. The electrical connection unit has an electrical connection port and a plurality of conducting lines electrically connected to the electrical connection port. The plurality of conducting lines is arranged on the first face of the pad. The plurality of retaining members is electrically connected to the plurality of conducting lines. Each of the plurality of retaining members includes a fixing portion. Each of the fixing portions of the plurality of retaining members forms a space in communication with a respective one of the plurality of through-holes, and is fixed to the pad. The plurality of electrode pieces is electrically connected to the plurality of retaining members. Each of the plurality of electrode pieces includes a connection portion and a detection portion. Each of the connection portions of the plurality of electrode pieces is engaged with the fixing portion of a respective one of the plurality of retaining members.

In a form shown, each of the plurality of retaining members includes a first fitting member and a second fitting member. The first fitting member includes a first cylindrical portion and a first extending portion. The first extending portion is located at a closed end of the first cylindrical portion. The second fitting member includes a second cylindrical portion and a second extending portion. The second extending portion is located at an open end of the second cylindrical portion. The second cylindrical portion of the second fitting member is received in the first cylindrical portion of the first fitting member. The first extending portion abuts with the first face of the pad via one of the plurality of conducting lines. The second extending portion abuts with the second face of the pad. The fixing portion is formed in the second cylindrical portion of the second fitting member.

In the form shown, the fixing portion is in a form of a recess having a first positioning portion. The connection portion includes a second positioning portion, and the first positioning portion is configured to fix the second positioning portion.

In the form shown, the detection portion includes a fixing plate, and one face of the fixing plate facing away from the pad includes an adhesive layer.

In the form shown, the electrocardiogram detector further includes a covering piece attached to the adhesive layers of the plurality of electrode pieces.

In the form shown, the electrocardiogram detector further includes a protective layer arranged on the first face of the pad. The protective layer and the pad jointly cover the plurality of conducting lines.

Based on this, since the electrocardiogram detector uses the retaining members to fix the electrode pieces to the pad, the electrocardiogram detector of the disclosure does not need to dispose the electrode pieces one by one. Advantageously, convenient operation of the electrocardiogram detector can be attained.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1 shows a conventional electrocardiogram detector.

FIG. 2 shows an electrocardiogram detector according to a first embodiment of the disclosure wherein the retaining member and the electrode piece are separate from the pad.

FIG. 3 shows the electrocardiogram detector of the first embodiment of the disclosure wherein the retaining member and the electrode piece are attached to the pad.

FIG. 4 is a cross sectional view of the electrocardiogram detector of the first embodiment of the disclosure.

FIG. 5 is another cross sectional view of the electrocardiogram detector of the first embodiment of the disclosure where a protective layer is provided.

FIG. 6 is a cross sectional view of an electrocardiogram detector according to a second embodiment of the disclosure.

FIG. 7 is another cross sectional view of the electrocardiogram detector of the second embodiment of the disclosure where a protective layer is provided.

FIG. 8 shows an electrocardiogram detector electrically connected to an electrocardiograph according to the disclosure.

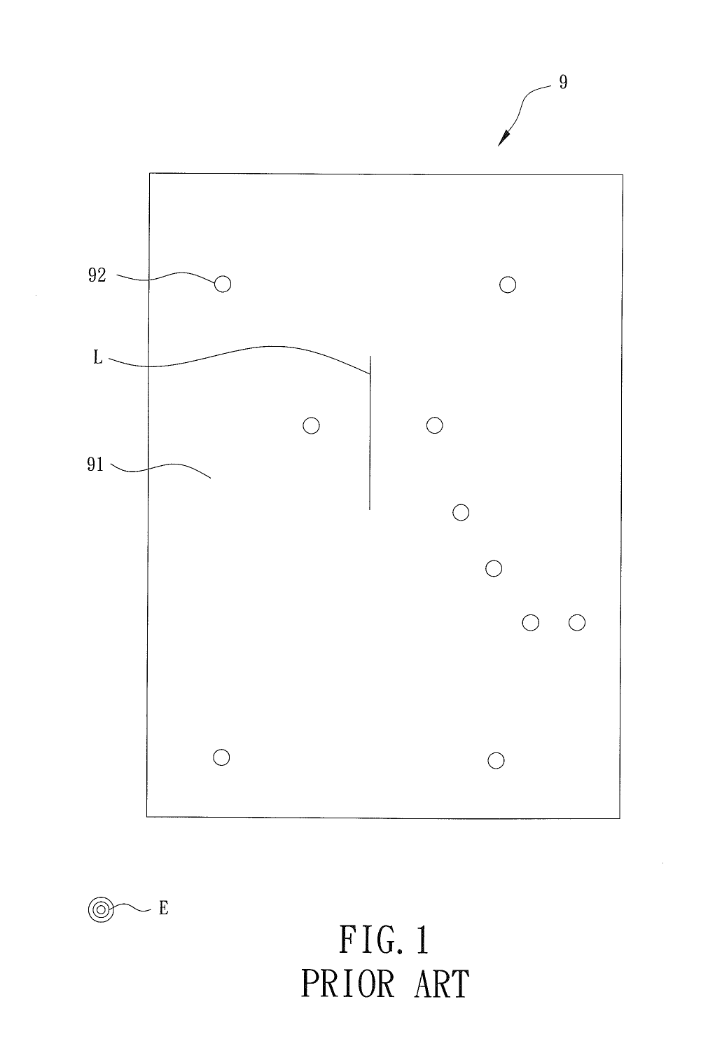

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

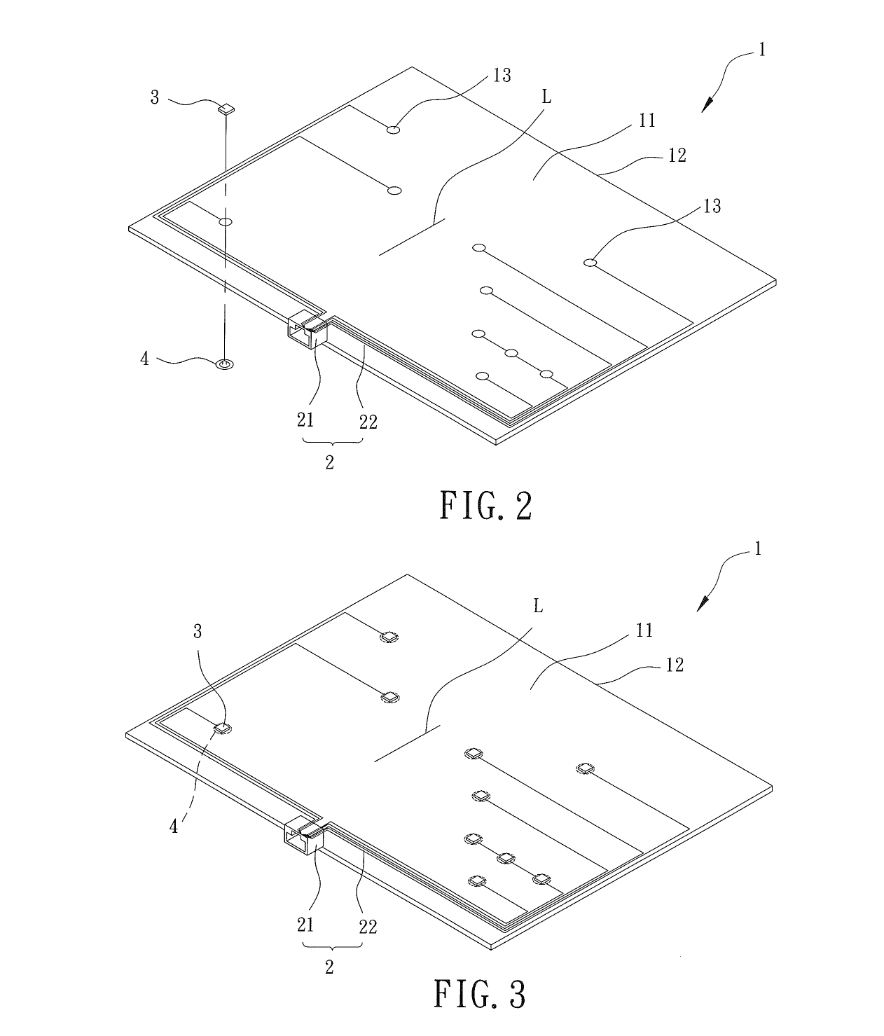

FIG. 2 shows an electrocardiogram detector according to a first embodiment of the disclosure. The electrocardiogram detector includes a pad 1, an electrical connection unit 2, a plurality of retaining members 3 and a plurality of electrode pieces 4. The electrical connection unit 2 is arranged on the pad 1. The retaining members 3 and the electrode pieces 4 are coupled to each other and fixed to the pad 1.

Referring to FIGS. 2 and 3, the pad 1 has a first face 11 and a second face 12. The pad 1 also has a plurality of through-holes 13 extending through the first face 11 and the second face 12. The pad 1 can be made of any material. In the embodiment, the pad 1 is made of a light-permeable, elastic material. As such, when the pad 1 is placed on the body of the user, the exact position of the pad 1 on the body of the user can be easily observed. The elasticity of the pad 1 permits the pad 1 to deform in any shape confirming to the body part of the user, increasing its utility. The quantity of the through-holes 13 is not limited, and the locations of the through-holes 13 are also not limited. In the embodiment, the through-holes 13 are arranged according to the detection locations of the twelve-lead electrocardiogram, and the quantity of the through-holes 13 is 10. The pad 1 can have any reference marking used to reflect the position of the pad 1, such as the line marking L as shown in the first embodiment. When the pad is placed on the chest of the user, the line marking L can be aligned with the midsternal line of the user. As such, the pad 1 can be properly placed on the body part of the user, increasing its utility.

The electrical connection unit 2 includes an electrical connection port 21 and a plurality of conducting lines 22 connected to the electrical connection port 21. The conducting lines 22 are arranged on the first face 11 of the pad 1. The conducting lines 22 may be formed on the first face 11 of the pad 1 by electroplating or printing process, so as to avoid the mutual interference and large space occupation of the conducting lines 22. Thus, the detection of the ECG signals is smooth and the utility is improved. Each conducting line 22 connects to the electrical connection port 21 at one end, extends to the through-hole 13 along the first face 11, and connects to the through-hole 13 at another end thereof. In this arrangement, when the retaining members 3 are coupled with the peripheries of the through-hole 13, the retaining members 3 can be electrically connected to the conducting lines 22. In this arrangement, the ECG detection can be carried out, increasing the utility thereof.

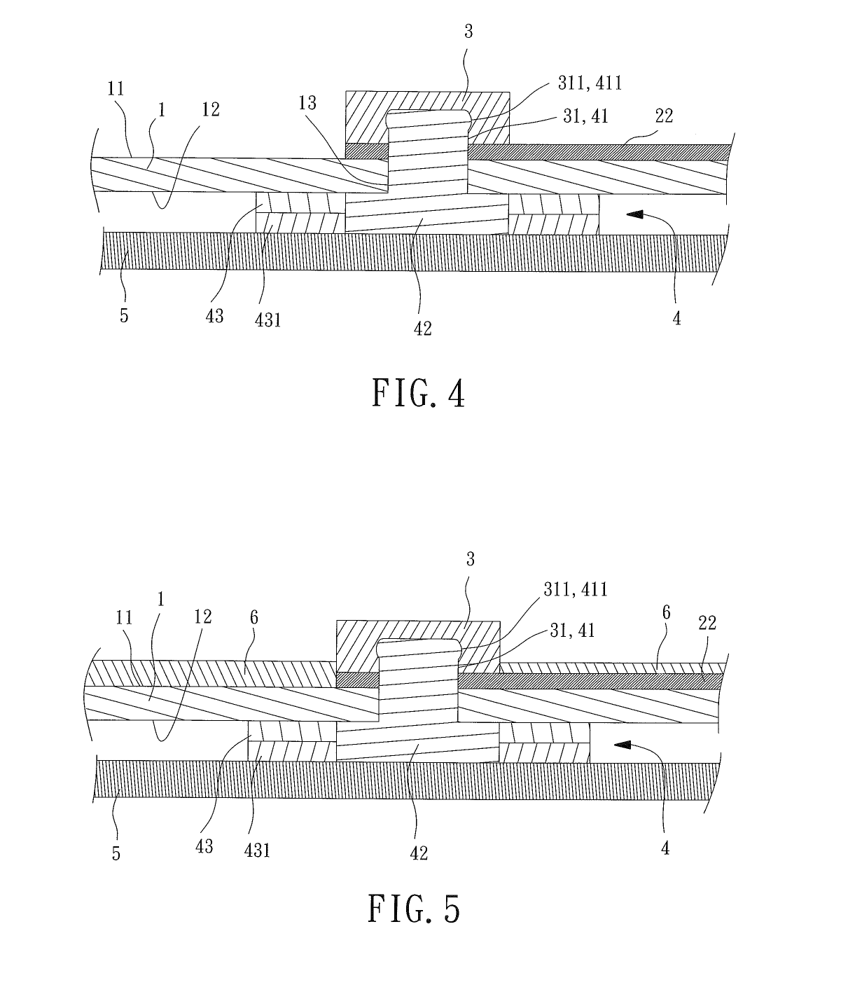

Referring to FIG. 4, in the first embodiment of the disclosure, each retaining member 3 is integrally formed and is made of an electrically conductive material. Each retaining member 3 includes a fixing portion 31 and is fixed to the first face 11 of the pad 1. The fixing portion 31 of each retaining member 3 forms a space in communication with a respective through-hole 13. The retaining members 3 are electrically connected to the conducting lines 22. The fixing portion 31 is in the form of a recess having a first positioning portion 311. The electrode piece 4 can be fixed in place by the first positioning portion 311. The retaining members 3 can be adhered to the first face 11 of the pad 1. In such an arrangement, when the first face 11 of the pad 1 is provided with the retaining members 3, the retaining members 3 can be electrically connected to the conducting lines 22 when the retaining members 3 are in contact with the conducting lines 22. In addition, when the retaining members 3 are used to fix the electrode pieces 4 in place, the ECG detection can be smoothly performed, thus increasing the utility of the electrocardiogram detector.

Each electrode piece 4 includes a connection portion 41 and a detection portion 42. The connection portion 41 of each electrode piece 4 extends through a respective through-hole 13 and is engaged with the fixing portion 31 of a respective retaining member 3. The electrode pieces 4 are electrically connected to the retaining members 3.

Specifically, the connection portion 41 of each electrode piece 4 may include a second positioning portion 411. When the second positioning portion 411 of the electrode piece 4 is engaged with the first positioning portion 311 of the fixing portion 31, the electrode piece 4 can be fixed in place by the retaining member 3. The first positioning portion 311 and the second positioning portion 411 are of any structures that can be engaged with each other. In the first embodiment, the first positioning portion 311 is a concave structure, and the second positioning portion 411 is a convex structure. Since the retaining member 3 is made of an electrically conductive material, when the connection portion 41 of the electrode piece 4 is engaged with the fixing portion 31 of the retaining member 3, the electrode piece 4 can be electrically connected to the retaining member 3. As a result, the electrode pieces 4 can be in electrical connection to the conducting lines 22. When the pad 1 is placed on the body of the user, the electrode pieces 4 can be in contact with the body of the user, permitting the ECG detection of the user. Thus, the utility is improved.

Furthermore, the detection portion 42 of each electrode piece 4 may further include a fixing plate 43. One face of the fixing plate 43 facing away from the pad 1 includes an adhesive layer 431. When the pad 1 is placed on the body of the user, the adhesive layer 431 can be adhered to the skin of the user, permitting the electrode pieces 4 to be fixed to the body of the user. Therefore, the utility is improved.

Moreover, the electrocardiogram detector according to the embodiment of the disclosure may further include a covering piece 5 attached to the adhesive layers 431 of the electrode pieces 4. The covering pieces 5 can be torn off when it is desired to use the adhesive layers 431. As such, the viscidity of the adhesive layers 431 can be maintained. The arrangement of the covering piece 5 is not limited herein. For example, the adhesive layer 431 of each electrode piece 4 can be attached by a covering piece 5. In this arrangement, according to different requirements, only those covering pieces 5 whose adhesive layers 431 are needed for use can be torn off. This can prevent the unused electrode pieces 4 from being adhered to the skin of the user via the adhesive layers 431 thereof, providing a flexible use of the electrocardiogram detector. In another option, the adhesive layers 431 of the electrode pieces 4 can be coupled with just a single covering piece 5. As such, when the covering piece 5 is torn off, each adhesive layer 431 can be exposed for use, permitting all of the electrode pieces 4 to be adhered to the skin of the user at once. In this case, it takes a shorter time to tear off one covering piece 5 as compared with the case of multiple covering pieces 5, improving the utility thereof.

Referring to FIG. 5, the electrocardiogram detector according to the first embodiment of the disclosure may further include a protective layer 6. The protective layer 6 is arranged on the first face 11 of the pad 1. The protective layer 6 and the pad 1 jointly cover the conducting lines 22. The protective layer 6 and the pad 1 may be made of the same or different materials. The protective layer 6 can prevent the exposure of the conducting lines 22, so that the conducting lines 22 can be protected from damage or deformation caused by an external force. Therefore, the electrical conductivity of the conducting lines 22 can be maintained.

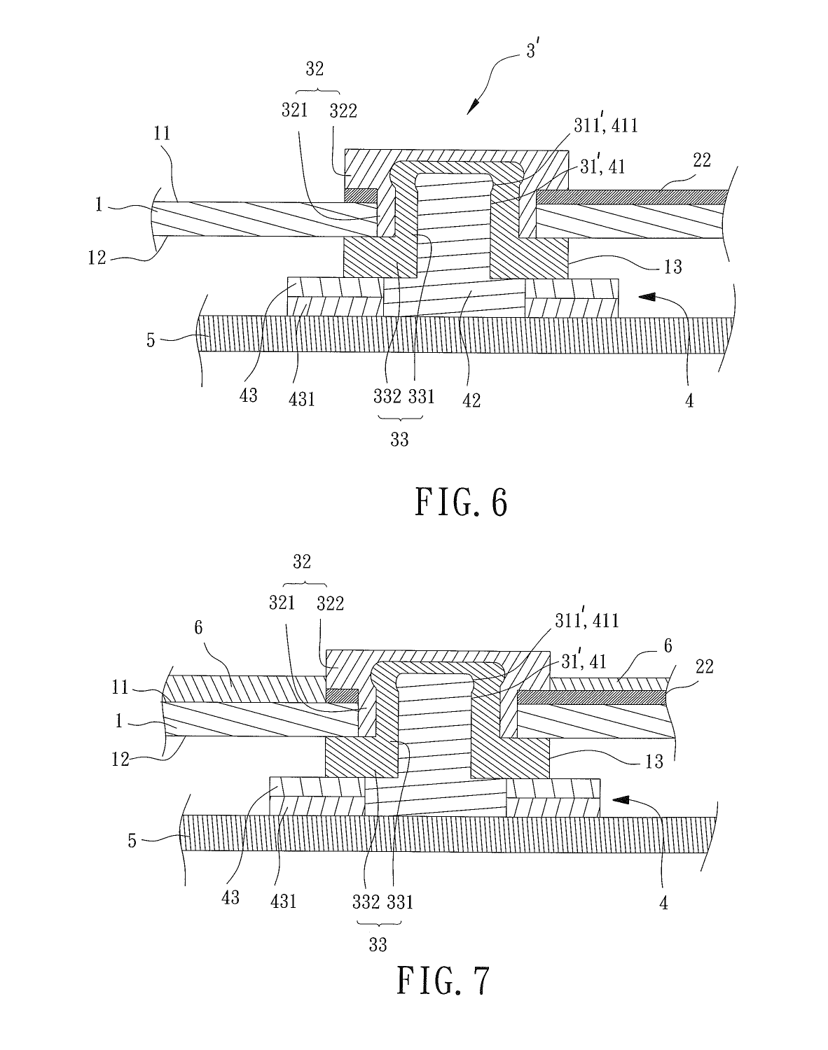

FIG. 6 shows an electrocardiogram detector according to a second embodiment of the disclosure. As compared with the above first embodiment, the electrocardiogram detector in the second embodiment includes a plurality of retaining members 3′, in which each retaining member 3′ is not integrally formed.

In the second embodiment, each retaining member 3′ includes a fixing portion 31′. The fixing portion 31′ includes a first positioning portion 311′. Each retaining member 3′ extends through a respective through-hole 13 and is fixed to the pad 1. The retaining members 3′ are electrically connected to the conducting lines 22. The connection portion 41 of the electrode piece 4 is engaged with the fixing portion 31′ of the retaining member 3′, and the second positioning portion 411 is fixed to the first positioning portion 311′.

Specifically, each retaining member 3′ includes a first fitting member 32 and a second fitting member 33. The first fitting member 32 includes a first cylindrical portion 321 and a first extending portion 322. The first extending portion 322 is located at a closed end of the first cylindrical portion 321. The second fitting member 33 includes a second cylindrical portion 331 and a second extending portion 332. The second extending portion 332 is located at an open end of the second cylindrical portion 331. The second cylindrical portion 331 of the second fitting member 33 is received in the first cylindrical portion 321 of the first fitting member 32. The first extending portion 322 abuts with the first face 11 of the pad 1 via the conducting line 22, and the second extending portion 332 abuts with the second face 12 of the pad 1. The first extending portion 322 is electrically connected to the conducting lines 22. The fixing portion 31′ is formed in the second cylindrical portion 331 of the second fitting member 33. The second cylindrical portion 331 of the second fitting member 33 can be received in the first cylindrical portion 321 of the first fitting member 32 in a press fitting manner. Alternatively, the first cylindrical portion 321 of the first fitting member 32 and the second cylindrical portion 331 of the second fitting member 33 can be engaged with each other via a tongue and groove structure. In such an arrangement, when the retaining members 3′ extend through the through-holes 13, the first extending portion 322 and the second extending portion 332 can prevent the disengagement of the retaining member 3′ from the through-hole 13. Thus, the positioning effect of the retaining members 3′ can be ensured. The first fitting member 32 and the second fitting member 33 are made of electrically conductive material. When the first fitting member 32 and the second fitting member 33 are combined with each other, the retaining member 3′ can provide an electrically conductive effect.

Referring to FIG. 7, the electrocardiogram detector of the second embodiment of the disclosure may also include the covering piece 5. The arrangement and shape of the covering piece 5 in the second embodiment is similar to what has been described in the first embodiment, and therefore is not discussed herein again.

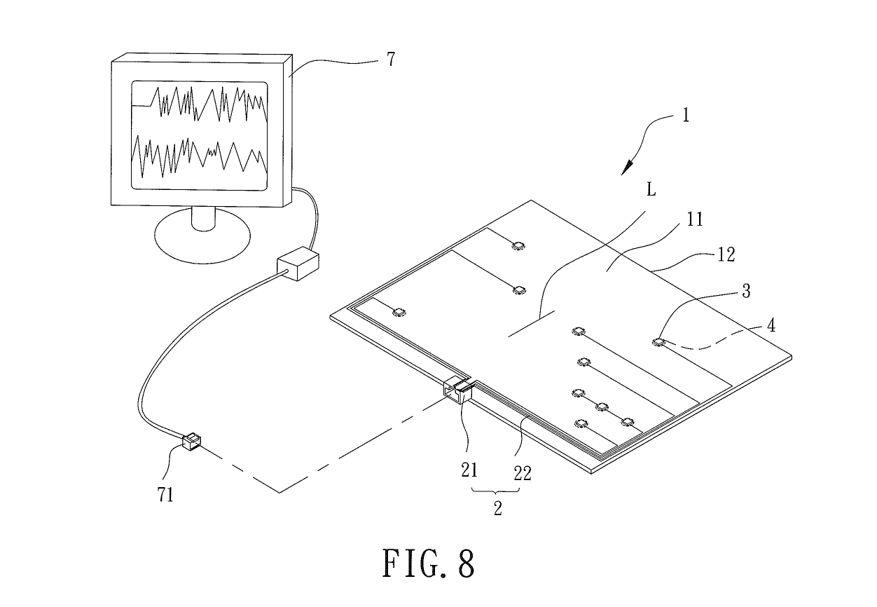

Referring to FIG. 8, the electrocardiogram detector of the disclosure may be used with an electrocardiograph 7. The electrocardiograph 7 can perform an electrocardiogram detection and generate an electrocardiogram of the testee. For example, the electrocardiograph 7 may be connected to the electrical connection port 21 via a wire 71, permitting the electrocardiograph 7 to electrically connect to the electrode pieces 4. In this arrangement, when the pad 1 is placed on the body of the user, the electrode pieces 4 can be in contact with the skin of the testee. Thus, the ECG information of the testee can be detected via the electrode pieces 4. The detected ECG information can be sent to the electrocardiograph 7 via the electrical connection unit 2. As a result, the ECG detection is completed, and the corresponding electrocardiogram is generated.

In summary, since the electrocardiogram detector uses the retaining members 3 to fix the electrode pieces 4 to the pad 1, the electrocardiogram detector of the disclosure can be directly used to detect the ECG signals of the testee without having to dispose the electrode pieces 4 one by one. Advantageously, convenient operation of the electrocardiogram detector can be attained.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. An electrocardiogram detector comprising:
a pad having a first face and a second face, wherein the pad comprises a plurality of through-holes extending through the pad from the first face to the second face;
an electrical connection unit having an electrical connection port and a plurality of conducting lines electrically connected to the electrical connection port, wherein the plurality of conducting lines is arranged on the first face of the pad;
a plurality of retaining members fixed to the first face of the pad and electrically connected to the plurality of conducting lines, wherein each of the plurality of retaining members comprises a fixing portion, and wherein each of the fixing portions of the plurality of retaining members forms a space in communication with a respective one of the plurality of through-holes; and
a plurality of electrode pieces electrically connected to the plurality of retaining members, wherein each of the plurality of electrode pieces comprises a connection portion and a detection portion, wherein each of the connection portions of the plurality of electrode pieces extends through a respective one of the plurality of through-holes and is engaged with the fixing portion of a respective one of the plurality of retaining members.

2. The electrocardiogram detector as claimed in claim 1, wherein the fixing portion is in a form of a recess having a first positioning portion, wherein the connection portion comprises a second positioning portion, wherein the first positioning portion is configured to fix the second positioning portion.

3. The electrocardiogram detector as claimed in claim 1, wherein the detection portion comprises a fixing plate, and one face of the fixing plate facing away from the pad comprises an adhesive layer.

4. The electrocardiogram detector as claimed in claim 3, further comprising a covering piece attached to the adhesive layers of the plurality of electrode pieces.

5. The electrocardiogram detector as claimed in claim 1, further comprising a protective layer arranged on the first face of the pad, wherein the protective layer and the pad jointly cover the plurality of conducting lines.

6. An electrocardiogram detector comprising:
a pad having a first face and a second face, wherein the pad comprises a plurality of through-holes extending through the pad from the first face to the second face;
an electrical connection unit having an electrical connection port and a plurality of conducting lines electrically connected to the electrical connection port, wherein the plurality of conducting lines is arranged on the first face of the pad;
a plurality of retaining members electrically connected to the plurality of conducting lines, wherein each of the plurality of retaining members comprises a fixing portion, wherein each of the fixing portions of the plurality of retaining members forms a space in communication with a respective one of the plurality of through-holes, and is fixed to the pad; and
a plurality of electrode pieces electrically connected to the plurality of retaining members, wherein each of the plurality of electrode pieces comprises a connection portion and a detection portion, wherein each of the connection portions of the plurality of electrode pieces is engaged with the fixing portion of a respective one of the plurality of retaining members.

7. The electrocardiogram detector as claimed in claim 6, wherein each of the plurality of retaining members comprises a first fitting member and a second fitting member, wherein the first fitting member comprises a first cylindrical portion and a first extending portion, wherein the first extending portion is located at a closed end of the first cylindrical portion, wherein the second fitting member comprises a second cylindrical portion and a second extending portion, wherein the second extending portion is located at an open end of the second cylindrical portion, wherein the second cylindrical portion of the second fitting member is received in the first cylindrical portion of the first fitting member, wherein the first extending portion abuts with the first face of the pad via one of the plurality of conducting lines, wherein the second extending portion abuts with the second face of the pad, and wherein the fixing portion is formed in the second cylindrical portion of the second fitting member.

8. The electrocardiogram detector as claimed in claim 6, wherein the fixing portion is in a form of a recess having a first positioning portion, wherein the connection portion comprises a second positioning portion, wherein the first positioning portion is configured to fix the second positioning portion.

9. The electrocardiogram detector as claimed in claim 7, wherein the fixing portion is in a form of a recess having a first positioning portion, wherein the connection portion comprises a second positioning portion, wherein the first positioning portion is configured to fix the second positioning portion.

10. The electrocardiogram detector as claimed in claim 6, wherein the detection portion comprises a fixing plate, and one face of the fixing plate facing away from the pad comprises an adhesive layer.

11. The electrocardiogram detector as claimed in claim 10, further comprising a covering piece attached to the adhesive layers of the plurality of electrode pieces.

12. The electrocardiogram detector as claimed in claim 6, further comprising a protective layer arranged on the first face of the pad, wherein the protective layer and the pad jointly cover the plurality of conducting lines.
